(19) United States
(12) Patent Application Publication
(10) Pub. No.: US 2008/0214808 A1
(43) Pub. Date: Sep. 4, 2008

(54) Preparation of Aseptic 3-[2-[4-((6-Fluoro-1,2-Benzisoxazol-3-Yl)-1-Piperidinyl]-6,7,8,9-Tetrahydro-9-Hydroxy-2-Methyl-4H-Pyrido[1,2-a]Pyrimidin-4-One Palmitate Ester

(76) Inventors: Thomas Frederik Ernestine Spittaels, Antwerpen (BE); Joannes Petrus Van Dun, Lille (BE); Jurgen Alois Verbraeken, Vosselaar (BE); Benny Wouters, Herentals (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/912,452

(22) PCT Filed: Apr. 20, 2006
(86) PCT No.: PCT/EP06/61694

§ 371 Date: Oct. 24, 2007

(30) Foreign Application Priority Data
Apr. 25, 2005 (EP) 05103343.9
Apr. 26, 2005 (EP) 05103391.8

(51) Int. Cl. C07D 239/70

(52) U.S. Cl. 544/282

(57) ABSTRACT

The present invention concerns a process for preparing aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3 -yl)-1-piperidinyl]ethyl]-6,7-dihydro-2-methyl-4H-pyrido[1,2-a]-pyrimidin-4-one (II-b), and 3-[2-[4-(6-fluoro-1,2-benzisoxazol-3-yl)-1-piperidinyl]-ethyl]-6,7,8,9-tetrahydro-2-methyl-9-pentadecyl-4H-pyrido[1,2-a]pyrimidin-4-one (III), and having an average particle size ranging from 20 to 150 μm.

### BACKGROUND OF THE INVENTION

The present invention concerns a process for preparing aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b), and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), and having an average particle size ranging from 20 to 150 μm, preferably from 20 to 80 μm. 3-[2-[4-(6-Fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) is also known as paliperidone palmitate ester; and the compound of formula (II-a) is also known as paliperidone.

In EP-0,368,388 (U.S. Pat. No. 5,158,952), 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester of formula (I) is disclosed.

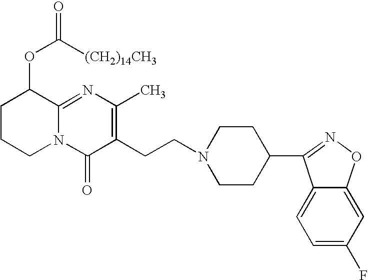

EP-0,904,081 and EP-1,033,987 disclose aqueous suspensions of ‘submicron’ paliperidone palmitate (I) suitable as depot formulations which are therapeutically effective for about a month when administered intramuscularly to a warm-blooded subject. During pharmaceutical development, aseptic formulations of paliperidone palmitate (I) were initially obtained by gamma irradiation. Upon analysis of irradiated paliperidone (I), the process was found to give three breakdown products: up to 0.24% of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a) and 3-[2-[4-(6-fluoro-1,2-benzisoxazol-3-yl)-1-piperidinyl]ethyl]-6,7-dihydro-2-methyl-4H-pyrido[1,2-a]-pyrimidin-4-one (1-b) which in the analytical HPLC method co-eluted and are collectively designated (II) hereinafter,

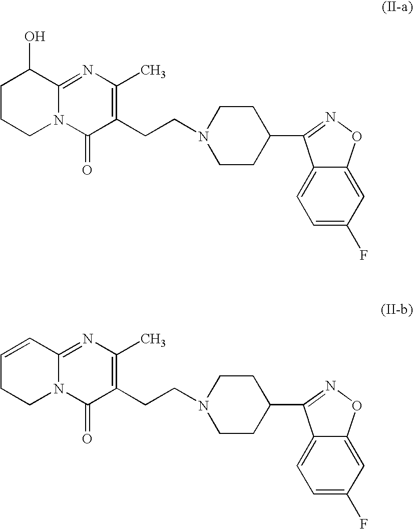

and up to 0.46% of 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III).

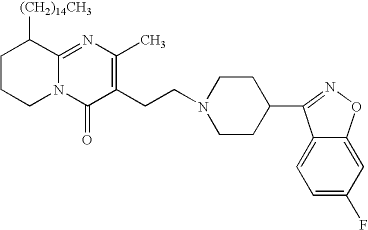

In order to avoid the formation of the breakdown products (II) [i.e. (II-a) and (1-b)] and (III), various other techniques to sterilize compound (I) were considered. Sterilization by microfiltration is impossible because the aqueous suspension of ‘submicron’ paliperidone palmitate (I) will block the filter pores. Heat sterilization proves impossible as compound (I) melts between 116.5 and 119.5° C.

The double objective of developing an aseptic production process for paliperidone palmitate (I) while managing its particle size distribution is achieved in the present invention which provides a process for preparing aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester of formula (I)

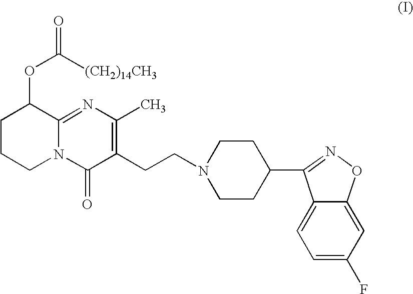

substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b) and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), and having an average particle size ranging from 20 to 150 μm, preferably from 20 to 80 μm, comprising the steps of
a) heating 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (1) and ethanol parenteral grade to 72° C. to 78° C.;
b) filtering the solution of step a) over a sterile 0.22 μm filter into a sterile crystallization reactor;
c) allowing 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) to crystallize while cooling; and either
d) filtering off the thus obtained crystals; or
e) reheating the thus obtained suspension again to 72° C. to 78° C.;
f) allowing 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) to crystallize while cooling; and
g) filtering off the thus obtained crystals.

The terms ‘aseptic’ and ‘sterile’ are used herein interchangeably and mean ‘free or freed from micro-organisms’. All process steps following step b) are conducted aseptically under fully closed conditions applying isolator technology.

The process comprising the steps a), b), c), e), f) and g), that is the process comprising two heating cycles, is the more robust one as it allows the best control over the crystallization process and the particle size distribution of the particles.

The temperature achieved in step e) and the rate of cooling applied in step f) are particular important to the particle size distribution of aseptic paliperidone palmitate ester (I). Reheating to just below reflux temperature (<77° C.) and cooling at a rate of 0.5° C./min yields crystals having an average particle size of about 80 micron. Reheating to just below reflux temperature (<77° C.) and cooling at a rate of 1° C./min yields crystals having an average particle size of about 50 to 60 micron. In both instances, crystallization starts at about 60° C. These conditions and parameters are equipment specific (here for a 30 L reactor) and may vary when larger equipment is used.

Reheating to reflux temperature (78° C.) and rapid cooling yields crystals having an average particle size of about 20 to 30 micron. It is preferable that the rate of cooling in step f) is as rapid as possible.

Notwithstanding the aforementioned, a process comprising the steps a), b), c) and d), that is a process comprising only one heating cycle, is also feasible as can be seen from particular experiments in the experimental part.

In a further aspect of the invention, there is provided a process as described hereinbefore, comprising the further steps of

h) suspending the crystals obtained in steps d) or g) in a sterilized solution of water comprising a surfactant, and optionally a suspending agent and a buffer;
i) grinding the suspension of step h) in the presence of a grinding medium to particles having a specific surface area >4 m²/g;
j) sieving the suspension of step i) to remove the grinding medium;
k) diluting and mixing the solution of step j) with a sterilized solution of water optionally comprising a suspending agent, a buffer and an antioxidant; and
l) filling the sieved suspension into a sterile container.

These further process steps are known from EP-0,904,081 and EP-1,033,987. In particular, the sterilized solution of water comprising a surfactant, and optionally a suspending agent and a buffer is prepared by dissolving a surfactant, and optionally a suspending agent and a buffer in water for injection and sterilizing the thus obtained solution by heating for 30 minutes at 121° C., or by microfiltration. The grinding process is a wet milling process as disclosed in EP-0,499,299.

The particles of the present invention have a surfactant or surface modifier adsorbed on the surface thereof in an amount sufficient to maintain a specific surface area >4 m²/g (i.e. corresponding to an average particle size of less than 2,000 nm), preferably the specific surface area >6 m²/g, and in particular is in the range from 10 to 16 m²/g. Useful surface modifiers are believed to include those which physically adhere to the surface of the active agent but do not chemically bond thereto.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants. Representative examples of excipients include gelatin, casein, lecithin (phosphatides), gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene allyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminate silicate, triethanolamine, polyvinyl alcohol (PVA), poloxamers, tyloxapol and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986. The surface modifiers are commercially available and/or can be prepared by techniques known in the art. Two or more surface modifiers can be used in combination.

Particularly preferred surface modifiers include polyvinylpyrrolidone; tyloxapol; poloxamers, such as Pluronic™ F68, F108 and F127 which are block copolymers of ethylene oxide and propylene oxide available from BASF; poloxamines, such as Tetronicm™ 908 (T908) which is a tetrafunctional block copolymer derived from sequential addition of ethylene oxide and propylene oxide to ethylenediamine available from BASF; dextran; lecithin; Aerosol OT™ (AOT) which is a dioctyl ester of sodium sulfosuccinic acid available from Cytec Industries; Duponol™ P which is a sodium lauryl sulfate available from DuPont; Triton™ X-200 which is an alkyl aryl polyether sulfonate available from Rohm and Haas; Tweens™ 20, 40, 60 and 80 which are polyoxyethylene sorbitan fatty acid esters available from ICI Speciality Chemicals; Span™ 20, 40, 60 and 80 which are sorbitan esters of fatty acids; Arlacel™ 20, 40, 60 and 80 which are sorbitan esters of fatty acids available from Hercules, Inc.; Carbowax™ 3550 and 934 which are polyethylene glycols available from Union Carbide; Crodesta™ F 110 which is a mixture of sucrose stearate and sucrose distearate available from Croda Inc.; Crodesta™ SL-40 which is available from Croda, Inc.; hexyldecyl trimethyl ammonium chloride (CTAC); bovine serum albumin and SA90HCO which is C₁₈H₁₇CH₂ (CON(CH₃)CH₂(CHOH)₄CH₂OH)₂. The surface modifiers which have been found to be particularly useful include tyloxapol and a poloxamer, preferably, Pluronic™ F108 and Pluronic™ F68, and polyoxyethylene sorbitan fatty acid esters, preferably Tween™ 20.

Pluronic™ F108 corresponds to poloxamer 338 and is the polyoxyethylene, polyoxypropylene block copolymer that conforms generally to the formula HO[CH₂CH₂O]ₓ[CH(CH₃)CH₂O]_{y}[CH₂CH₂O)_{z}H in which the average values of x, y and z are respectively 128, 54 and 128. Other commercial names of poloxamer 338 are Hodag Nonionic™ 1108-F available from Hodag, and Synperonic™ PE/F108 available from ICI Americas.

The optimal relative amount of paliperidone palmitate and the surface modifier depends on various parameters. The optimal amount of the surface modifier can depend, for example, upon the particular surface modifier selected, the critical micelle concentration of the surface modifier if it forms micelles, the surface area of (I), etc. The specific surface modifier preferably is present in an amount of 0.1 to 1 mg per square meter surface area of (I). In case Pluronic™ F108 is used as a surface modifier, a ratio (w/w) of (I): surface modifier of approximately 6:1 is preferred. When Tween™ 20 is the surface modifier, a ratio (w/w) of (I): surface modifier of approximately 13:1 is preferred.

As used herein, an effective average particle size of less than 2,000 nm means that at least 90% of the particles have a diameter of less than 2,000 nm when measured by art-known conventional techniques, such as sedimentation field flow fractionation, photon correlation spectroscopy or disk centrifugation. With reference to the effective average particle size, it is preferred that at least 95% and, more preferably, at least 99% of the particles have a particle size of less than the effective average particle size, e.g. 2,000 nm. Most preferably, essentially all of the particles have a size of less than 2,000 nm.

The grinding media for the particle size reduction step can be selected from rigid media preferably spherical or particulate in form having an average size less than 3 mm and, more preferably, less than 1 mm. Such media desirably can provide the particles of the invention with shorter processing times and impart less wear to the milling equipment. The selection of the material for the grinding media is believed not to be critical. However, 95% ZrO stabilized with magnesia, zirconium silicate, and glass grinding media provide particles having levels of contamination which are believed to be acceptable for the preparation of pharmaceutical compositions. Further, other media, such as polymeric beads, stainless steel, titania, alumina and 95% ZrO stabilized with yttrium, are useful. Preferred grinding media have a density greater than 2.5 g/cm³ and include 95% ZrO stabilized with magnesia and polymeric beads.

The attrition time can vary widely and depends primarily upon the particular mechanical means and processing conditions selected.

The particles must be reduced in size at a temperature which does not significantly degrade the antipsychotic agent. Processing temperatures of less than 30 to 40° C. are ordinarily preferred. If desired, the processing equipment may be cooled with conventional cooling equipment. The method is conveniently carried out under conditions of ambient temperature and at processing pressures which are safe and effective for the milling process.

Aqueous compositions according to the present invention conveniently further comprise a suspending agent, a buffer and an antioxidant. Particular ingredients may function as two or more of these agents simultaneously, e.g. behave like a preservative and a buffer, or behave like a buffer and an isotonizing agent, or like a buffering agent and antioxidant.

Suitable suspending agents for use in the aqueous suspensions according to the present invention are cellulose derivatives, e.g. methyl cellulose, sodium carboxymethyl cellulose and hydroxypropyl methyl cellulose, polyvinylpyrrolidone, alginates, chitosan, dextrans, gelatin, polyethylene glycols, polyoxyethylene- and polyoxy-propylene ethers. Preferably sodium carboxymethyl cellulose is used in a concentration of 0.5 to 2%, most preferably 1% (w/v). Suitable wetting agents for use in the aqueous suspensions according to the present invention are polyoxyethylene derivatives of sorbitan esters, e.g. polysorbate 20 and polysorbate 80, lecithin, polyoxyethylene- and polyoxypropylene ethers, sodium deoxycholate. Preferably polysorbate 20 is used in a concentration of 0.5 to 3%, more preferably 0.5 to 2%, most preferably 1.1% (w/v).

Suitable buffering agents are salts of weak acids and should be used in amount sufficient to render the dispersion neutral to very slightly basic (up to pH 8.5), preferably in the pH range of 7 to 7.5. Particularly preferred is the use of a mixture of disodium hydrogen phosphate (anhydrous) (typically about 0.9% (w/v)) and sodium dihydrogen phosphate monohydrate (typically about 0.6% (w/v)). This buffer also renders the dispersion isotonic and, in addition, less prone to flocculation of the ester suspended therein. Citric acid is useful as an antioxidant.

Suitable sterile containers in which the suspension of paliperidone palmitate ester (I) may be filled comprise sterile holding vessels as well sterile syringes which then may packaged with appropriate needles into end-user packages.

The present invention also concerns aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b), and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), and having an average particle size ranging from 20 to 80 μm.

More in particular, the invention relates to aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) containing less than 0.5% of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]-pyrimidin-4-one (II-b), and less than 0.01% of 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]-pyrimidin-4-one (III), and having an average particle size ranging from 20 to 80 μm.

Further, the invention concerns aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]-pyrimidin-4-one palmitate ester (I) substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b), and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), and having a specific surface area >4 m²/g.

### EXPERIMENTAL PART

### Comparative Example

Compound (I) was irradiated with various doses of gamma rays in different containers. The amount of the breakdown products (II) [i.e. the sum of the amounts of compound (II-a) and (II-b)] and (III) increased dose-dependently.

| Container | Dose (kGY) | (I) | (II) | (III) |
|---|---|---|---|---|
| Glass | 0 | 99.0 | — | — |
| | 5 | 98.8 | 0.02 | 0.08 |
| | 10 | 98.7 | 0.05 | 0.15 |
| | 15 | 98.5 | 0.11 | 0.23 |
| | 20 | 98.3 | 0.17 | 0.34 |
| | 25 | 98.2 | 0.18 | 0.36 |
| | 30 | 98.0 | 0.24 | 0.46 |
| Glass/metal | 0 | 99.0 | — | — |
| | 5 | 98.8 | 0.02 | 0.08 |
| | 10 | 98.9 | — | 0.10 |
| | 15 | 98.5 | 0.11 | 0.23 |
| | 20 | 98.4 | 0.15 | 0.29 |
| | 25 | 97.2 | 0.05 | 0.35 |
| | 30 | 98.2 | 0.21 | 0.45 |
| Plastic | 0 | 99.0 | — | — |
| | 15 | 98.3 | 0.03 | 0.23 |
| | 20 | 97.9 | 0.03 | 0.29 |
| | 25 | 97.2 | 0.06 | 0.35 |

### Example 1

### GMP Batches in Pilot Installation

All equipment was sterilized using the following techniques:
steam sterilization
dry heat sterilization
vaporized hydrogen peroxide (VHP) sterilization
gamma irradiation

To improve the sterility assurance of the process, all critical handlings with regard to sterility were performed in an isolator.

A reaction vessel was charged with 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (2.5 kg) and ethanol parenteral grade (7 L/kg) and heated to reflux temperature (78-79° C.) while stirring. The product dissolved at about 70° C. The solution was filtered at 76° C. over a sterile 0.22 μm filter into a glass crystallization reactor. The sterile filter was then washed with heated ethanol (1 L/kg).

The filtrate cooled to room temperature whereupon the product crystallized. The thus obtained suspension was either filtered off or reheated again.

Reheating to just below reflux temperature (<77° C.) and cooling at a rate of 0.5° C./min yielded crystals having an average particle size of about 80 micron. Reheating to just below reflux temperature (<77° C.) and cooling at a rate of 1° C./min yielded crystals having an average particle size of about 50 to 60 micron. In both instances, crystallization started at about 60° C.

Reheating to reflux temperature (78° C.) and rapid cooling yielded crystals having an average particle size of about 20 to 30 micron.

The crystals were then filtered off, washed with ethanol parenteral grade (1 L/kg) and dried in vacuo at 50° C. in Tyvek bags so as to prevent dust formation.

HPLC analyses showed that the amount of the compound (I) was 99.4% or more while the amount of (II-a) was 0.07% or lower and compounds (II-b) and (III) were not detectable in any of the samples.

8 Batches were run, yielding product with a particle size distribution measured by laser diffraction as shown in Table 1.

TABLE 1

| | | Crystallization | | | | | | Particle size | | | |
|---|---|---|---|---|---|---|---|---|---|---|---|
| | | Calculated | | start | | | | distribution | | | |
| | Cooling | cooling gradient | Tmax | cooling ° C. | | start at (° C.) | | dl10 | dl50 | dl90 | Yield |
| Run | rate | (° C./min) | Treactor | Tjacket | Treactor | Treactor | Tjacket | (μm) | (μm) | (μm) | (%) |
| 1 | | | | | | | | | | | |
| first crystallization | 1° C./min | 1.18 | 76 | 80 | 75.6 | 58 | 24.7 | na | na | na | |
| second crystallization | 1° C./min | 1.01 | 75 | 80 | 75 | 61 | 29.3 | 244 | 73 | 18 | 89.7 |
| 2 | | | | | | | | | | | |
| first crystallization | max | 1.13 | 78 | 80 | 76 | 58 | 22 | na | na | na | |
| second crystallization | max | 1.13 | 77.5 | 80 | 73 | 50 | 13 | 95 | 29 | 9 | 95.2 |
| 3 | | | | | | | | | | | |
| first crystallization | max | 1.01 | 76.5 | 80 | 75 | 57.6 | na | na | na | na | |
| second crystallization | max | 1.01 | 78 | 80 | 77.5 | 46.7 | na | 104 | 20 | 7 | 96.2 |
| 4 | | | | | | | | | | | |
| first crystallization | 1° C./min | 1.15 | 76.5 | 80 | 74 | 47 | 12.2 | na | na | na | |
| second crystallization | 1° C./min | 1.01 | 76 | 80 | 74 | 61.9 | 28.1 | 285 | 82 | 19 | 73.4 |
| 5 | | | | | | | | | | | |
| first crystallization | 1° C./min | 0.98 | 76 | 80 | 75 | 60.5 | 27.5 | 171 | 58 | 15 | 94.3 |
| second crystallization | 1° C./min | na | na | na | na | na | na | na | na | na | |
| 6 | | | | | | | | | | | |
| first crystallization | 1° C./min | 0.94 | 76 | 80 | 76 | 57 | 22 | 276 | 56 | 15 | 94.5 |
| second crystallization | 1° C./min | na | na | na | na | na | na | na | na | na | |
| 7 | | | | | | | | | | | |
| first crystallization | 1° C./min | 0.94 | 76 | 80 | 76 | 62 | 32 | 183 | 67 | 17 | 97.0 |
| second crystallization | 1° C./min | na | na | na | na | na | na | na | na | na | |
| 8 | | | | | | | | | | | |
| first crystallization | 1° C./min | 1.11 | 75.5 | 80 | 75 | 32 | −4 | na | na | na | |
| second crystallization | 1° C./min | 0.73 | 74 | 80 | 73 | 62 | 29 | 151 | 57 | 15 | 91.8 |

### Example 2

### Scale Up and Equipment Set Up in Hastelloy C22 Mini Plant Vessels of 30 L, 60 L and 160 L

A reactor was charged with 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]-pyrimidin-4-one palmitate ester and ethanol parenteral grade (8 L/kg) and heated to reflux temperature (78-79° C.) while stirring. The product dissolved at about 70° C.

The reaction mixture is then cooled to room temperature whereupon the product crystallized. The thus obtained suspension was reheated again. The solution was cooled using differing cooling gradients (in consecutive experiments, the mixture was reheated and cooled again; after each cooling gradient, a sample was taken and isolated using a filter. The particle characteristics were determined.

HPLC analyses showed that the amount of (II-a) was 0.1% or lower, and compounds (II-b) and (III) were not detectable in any of the samples.

Different batches were run, yielding product with a particle size distribution measured by laser diffraction as shown in Tables 2 to 4.

TABLE 2
30 L scale experiments

| | Crystallization | | | | | Particle | | |
|---|---|---|---|---|---|---|---|---|
| | Calculated | | | | | size distribution | | |
| | cooling gradient | Tmax | start at . . . (° C.) | | start cooling (° C.) | dl10 | dl50 | dl90 |
| Cooling rate | (° C./min) | Treactor | Treactor | Tjacket | Treactor | (μm) | (μm) | (μm) |
| 1° C./min | 1.04 | 79.7 | 25.8 | 24.7 | 79.6 | 647 | 12 | 3.6 |
| max | 8.95 | 77.5 | 56 | −1 | 75.6 | 145 | 32 | 8.5 |
| 1° C./min | 0.86 | 76.3 | 64.7 | 59.1 | 75.4 | 292 | 95 | 22 |
| 1° C./min | 0.82 | 76.6 | 65.1 | 59.1 | 75.4 | 279 | 98 | 21 |
| 0.7° C./min | 0.63 | 76.6 | 64.5 | 61 | 75.9 | 262 | 102 | 27 |
| 0.4° C./min | 0.36 | 76.3 | 64.8 | 61.6 | 75.7 | 345 | 107 | 26 |

TABLE 3
60 L scale experiments

| | Crystallization | | | | | Particle | | |
|---|---|---|---|---|---|---|---|---|
| | Calculated | | | | | size distribution | | |
| | cooling gradient | Tmax | start at . . . (° C.) | | start cooling (° C.) | dl10 | dl50 | dl90 |
| Cooling rate | (° C./min) | Treactor | Treactor | Tjacket | Treactor | (μm) | (μm) | (μm) |
| 0.4° C./min | 0.37 | 79.3 | 64.0 | 59.8 | 79.1 | 558.8 | 74.2 | 13.3 |
| 2.0° C./min | 1.42 | 79.6 | 60.4 | 44.5 | 75.0 | 805.3 | 44.4 | 9.3 |
| 0.7° C./min | 0.67 | 77.3 | 62.3 | 55.3 | 75.2 | 562.1 | 59.7 | 11.7 |
| 1.0° C./min | 0.81 | 78.9 | 61.9 | 52.3 | 74.9 | 562.7 | 52.0 | 10.6 |
| 1.0° C./min | 0.88 | 79.7 | 62.1 | 51.6 | 74.8 | 446.5 | 55.1 | 11.5 |

TABLE 4
160 L scale experiments

| | Crystallization | | | | | Particle | | |
|---|---|---|---|---|---|---|---|---|
| | Calculated | | | | | size distribution | | |
| | cooling gradient | Tmax | start at . . . (° C.) | | start cooling (° C.) | dl10 | dl50 | dl90 |
| Cooling rate | (° C./min) | Treactor | Treactor | Tjacket | Treactor | (μm) | (μm) | (μm) |
| 1.0° C./min | 1.0 | 78.6 | 60.1 | 42.4 | 78.4 | | | |
| max | 2.9 | 78.6 | 58.2 | 9 | 78.4 | 146 | 36 | 9.6 |
| max | 3.2 | 75.6 | 58 | 11 | 75.5 | 279 | 41 | 9.8 |
| 1.0° C./min | 0.76 | 75.7 | 60.5 | 43.5 | 75.5 | 204 | 64 | 15 |
| 0.7° C./min | 0.5 | 75.7 | 63 | 54 | 75.5 | 285 | 84 | 20 |
| 0.4° C./min | 0.4 | 75.6 | 62.9 | 56.3 | 75.3 | 303 | 85 | 17 |
| 1° C./min | 0.75 | 75.8 | 61.5 | 47.4 | 75.7 | 198 | 60 | 13 |

### Example 3

### Crystallization in Stainless Steel Reactor of 50 L

All equipment was sterilized using dry heat sterilization. A stainless steel reactor was charged with 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]-pyrimidin-4-one palmitate ester and ethanol parenteral grade (8 L/kg) and heated to reflux temperature (78-79° C.) while stirring. The product dissolved at about 70° C.

The solution was filtered at 76° C. over a sterile 0.22 μm filter into a sterile crystallization reactor. The sterile filter was then washed with heated ethanol (1 L/kg).

The filtrate was reheated to reflux and then cooled to room temperature whereupon the product crystallized. The thus obtained suspension was reheated again. The solution was cooled using differing cooling gradients (in consecutive experiments, the mixture was reheated and cooled again; after each cooling gradient, a sample was taken and isolated using a filter. The crystals were dried in vacuo at 50° C. in Tyvek bags so as to prevent dust formation and the particle characteristics were determined.

Different batches were run, yielding product with a particle size distribution measured by laser diffraction as shown in Table 5.

TABLE 5

| | Crystallization | | | | | Particle | | |
|---|---|---|---|---|---|---|---|---|
| | Calculated | | | | | size distribution | | |
| | cooling gradient | Tmax | start at . . . (° C.) | | start cooling (° C.) | dl10 | dl50 | dl90 |
| Cooling rate | (° C./min) | Treactor | Treactor | Tjacket | Treactor | (μm) | (μm) | (μm) |
| 1° C./min | 0.95 | 78 | 63.5 | 60.2 | 77.5 | 156 | 65 | 16 |
| ASAP | 3.2 | 75.7 | 61.2 | 17.5 | 75 | 119 | 36 | 9.2 |
| 0.5° C./min | 0.48 | 75.7 | 63.8 | 62.7 | 75 | 192 | 80 | 20 |
| 0.5° C./min | 0.48 | 75.7 | 63.8 | 62.7 | 75 | 189 | 81 | 23 |
| 0.7° C./min | 0.81 | 75.7 | 61.7 | 58.9 | 75 | 113 | 41 | 11 |
| 1° C./min | 0.92 | 75.7 | 62.1 | 54.9 | 75 | 128 | 52 | 13 |

### Example 4

### Preparation of Finished Form

### Composition

TABLE 6

| | Amount Required | | | |
|---|---|---|---|---|
| | | | Quantity | |
| Name | Per ml | | for 24 L | |
| Paliperidone palmitate (sterile grade) | 156 | mg | 3.744 | kg |
| Polysorbate 20 parenteral | 12 | mg | 288 | g |
| Citric acid monohydrate parenteral | 5 | mg | 120 | g |
| Disodium hydrogen phosphate anhydrous | 5 | mg | 120 | g |
| parenteral | | | | |
| Sodium dihydrogen phosphate monohydrate | 2.5 | mg | 60 | g |
| parenteral | | | | |
| Sodium Hydroxide all use | 2.84 | mg | 68 | g |
| Polyethylene Glycol 4000 parenteral | 30 | mg | 720 | g |
| Water for injections q.s. ad | 1000 | μl | 24 | L |

### Equipment

stainless steel (SS) containers
Grinding media (Zirconium beads)+stainless steel (SS) grinding chamber
0.2 μm filters
40 μm filter
Filling unit
Autoclave
Dry heat oven

### Manufacturing

Zirconium beads wear cleaned and rinsed using water for injections and then depyrogenised by dry heat (120 min at 260° C.). Water for injections was transferred into a SS container. Polysorbate 20 was added and dissolved by mixing. The solution was sterilized by filtration through a sterile 0.2 μm filter into a sterilized SS container. Paliperidone palmitate ester (sterile grade) as prepared in the previous examples was dispersed into the solution and mixed until homogeneous. The suspension was milled aseptically in the grinding chamber using Zirconium beads as grinding media until the required particle size was reached. The suspension was filtered aseptically through a 40 μm filter into a sterilized SS container

Water for injections was transferred into a SS container, citric acid monohydrate parenteral, disodium hydrogen phosphate anhydrous, sodium dihydrogen phosphate monohydrate, sodium hydroxide all use, polyethylene glycol 4000 were added and mixed until dissolved. This solution was sterilized by filtration through a sterile 0.2 μm filter and transferred aseptically into the suspension. The final suspension was mixed until homogeneous. The suspension was filled aseptically into sterile syringes. The target dose volume was between 0.25 ml and 1.50 ml depending on the dose needed.

TABLE 7

| Dose volume | Target limit | lower limit | upper limit |
|---|---|---|---|
| 0.25 ml-1.00 ml | identical to | target limit − (target | target limit × 1.05 |
| | dose volume | limit × 0.05) | |
| 1.25 ml-1.50 ml | identical to | target limit − (target | target limit × |
| | dose volume | limit × 0.025) | 1.025 |

### Sterilization

All aseptic manipulations and sterilization processes were carried out according to FDA and European regulatory guidelines.

### Apparatus

Sterilization was done by steam sterilization (F₀≧15) of following equipment:
SS containers
Zirconium beads+grinding chamber
0.2 μm filters
40 μm filter
filling pump

### Immediate Container

1 ml long transparent plastic (COC) syringe with luer lock.
rubber tip cap, FM257/2 dark grey
rubber plunger stopper, 1 ml long, 4023/50, Fluorotec B2-40
2.25 ml transparent plastic (COC) syringe with luer lock.
rubber tip cap, FM257/2 dark grey
rubber plunger stopper, 1-3 ml, 4023/50, Fluorotec B2-40

The empty syringes with pre-assembled tip-caps were sterilized by gamma-irradiation (dose ≧25 kGy). The rubber plunger stoppers were sterilized by means of steam sterilization (F₀≧15).

1. A process for preparing aseptic crystalline 3-[2-[4-(6-fluoro-1,2-benzisoxazol-3-yl)-1-piperidinyl]ethyl]-6,7,8,9-tetrahydro-9-hydroxy-2-methyl-4H-pyrido-[1,2-a]pyrimidin-4-one palmitate ester of formula (I) 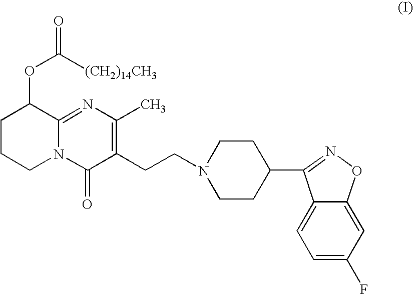
substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (1′-b), and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), having an average particle size ranging from 20 to 150 μm, preferably from 20 to 80 μm,
comprising the steps of
a) heating 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester (I) and ethanol parenteral grade to 72° C. to 78° C.;
b) filtering the solution over a sterile 0.22 μm filter into a sterile crystallization reactor;
c) allowing 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester to crystallize while cooling; and either
d) filtering off the thus obtained crystals; or
e) reheating the thus obtained suspension again to 72° C. to 78° C.;
f) allowing 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one palmitate ester to crystallize while cooling; and
g) filtering off the crystals.

2. The process according to claim 1 comprising the steps a), b), c), e), f) and g).

3. The process according to claim 1 wherein the reheating in step e) is to reflux temperature.

4. The process according to claim 3 wherein the cooling in step f) is conducted as rapidly as possible.

5. The process according to claim 1 wherein the reheating step e) is conducted at <77° C.

6. The process according to claim 1 comprising the steps a), b), c) and d).

7. The process according to claim 1 comprising the further steps of
h) suspending the crystals obtained in steps d) or g) in a sterilized solution of water comprising a surfactant, a suspending agent and a buffer;
i) grinding the suspension of step h) in the presence of a grinding medium to particles having a specific surface area >4 m²/g; j) sieving the suspension of step i) to remove the grinding medium; k) diluting and mixing the solution of step j) with a sterilized solution of water optionally comprising a suspending agent, a buffer and an antioxidant; and l) filling the sieved suspension into a sterile container.

8. Aseptic crystalline 3-[2-[4-(6-fluoro-1,2-benzisoxazol-3-yl)-1-piperidinyl]-ethyl]-6,7,8,9-tetrahydro-9-hydroxy-2-methyl-4H-pyrido[1,2-a]pyrimidin-4-one palmitate ester (I) substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b), and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), and having an average particle size ranging from 20 to 150 μm.

9. Aseptic crystalline 3-[2-[4-(6-fluoro-1,2-benzisoxazol-3-yl)-1-piperidinyl]-ethyl]-6,7,8,9-tetrahydro-9-hydroxy-2-methyl-4H-pyrido[1,2-a]pyrimidin-4-one palmitate ester (J) containing less than 0.5% of 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b), and less than 0.01% of 3-[2-[4-(6-fluoro-1,2-benzisoxazol-3-yl)-1-piperidinyl]ethyl]-6,7,8,9-tetrahydro-2-methyl-9-pentadecyl-4H-pyrido-[1,2-a]pyrimidin-4-one (III), and having an average particle size ranging from 20 to 150 μm.

10. Aseptic crystalline 3-[2-[4-(6-fluoro-1,2-benzisoxazol-3-yl)-1-piperidinyl]-ethyl]-6,7,8,9-tetrahydro-9-hydroxy-2-methyl-4H-pyrido[1,2-a]pyrimidin-4-one palmitate ester (I) substantially free of 3-[2-[4-(6-fluoro-1,2-benzisoxazol-3-yl)-1-piperidinyl]ethyl]-6,7,8,9-tetrahydro-9-hydroxy-2-methyl-4H-pyrido-[1,2-a]pyrimidin-4-one (II-a), 3-[2-[4-(6-fluoro-1,2-benzisoxazol-3-yl)-1-piperidinyl]ethyl]-6,7-dihydro-2-methyl-4H-pyrido[1,2-a]pyrimidin-4-one (II-b), and 3-[2-[4-(6-fluoro-1,2-benzisoxazol-3-yl)-1-piperidinyl]ethyl]-6,7,8,9-tetrahydro-2-methyl-9-pentadecyl-4H-pyrido[1,2-a]pyrimidin-4-one (III), and having a specific surface area >4 m²/g.
